(19) United States
(12) Patent Application Publication
(10) Pub. No.: US 2022/0062319 A1
(43) Pub. Date: Mar. 3, 2022

(54) COMBINATION IN THE TREATMENT OF NONTUBERCULOUS MYCOBACTERIAL DISEASES

(71) Applicant: Janssen Pharmaceutica NV, Beerse (BE)

(72) Inventors: Nacer LOUNIS, Wemmel (BE); Alexander Stephen PYM, Oxford (GB); Chrispin KAMBILI, Maplewood, NJ (US)

(73) Assignee: Janssen Pharmaceutica NV, Beerse (BE)

(21) Appl. No.: 17/421,546

(22) PCT Filed: Jan. 8, 2020
(86) PCT No.: PCT/EP2020/050247

§ 371 Date: Jul. 8, 2021

Related U.S. Application Data
(60) Provisional application No. 62/790,059, filed on Jan. 9, 2019.

(51) Int. Cl. A61K 31/7052; A61K 31/47; A61K 31/7048; A61K 31/133; A61P 31/04

(52) U.S. Cl. CPC A61K 31/7052; A61K 31/47; A61P 31/04; A61K 31/133; A61K 31/7048

(57) ABSTRACT

The present invention relates to a combination of bedaquiline, a macrolide (e.g. clarithromycin) and, optionally, ethambutol for use in the treatment of a disease associated with nontuberculous mycobacteria (NTM).

### FIELD OF THE INVENTION

The present invention relates to a combination for use in the treatment of nontuberculous mycobacteria, wherein the combination comprises bedaquiline (such as bedaquiline fumarate, marketed as Sirturo®), a macrolide (such as clarithromycin or azithromycin) and optionally another component for use in the treatment of nontuberculous mycobacteria (such as ethambutol). Other components that may also be a part of such combination include injectable aminoglycosides.

### BACKGROUND OF THE INVENTION

Nontuberculous mycobacterial (NTM) lung disease is a significant cause of morbidity and mortality among individuals with preexisting lung conditions such as bronchiectasis and COPD (chronic obstructive pulmonary disease).

Mycobacterium avium complex (MAC), Mycobacterium abscessus (Mab) and Mycobacterium kansasii are the mycobacterium species that result in NTM pulmonary disease (NTM-PD). NTM-PD is distinct from the pulmonary infection caused by Mycobacterium tuberculosis. Mycobacterium avium is part of the MAC that accounts for up to 70% of NTM-positive sputum cultures (although there are regional differences) and is one of the three NTM-PD species implicated in human disease. MAC are naturally-occurring organisms common in water and soil, often colonizing natural water sources such as indoor water systems, hot tubs and pools. MAC-pulmonary disease (MAC-PD) is most often seen in post-menopausal women and patients with underlying lung disease such as cystic fibrosis, bronchiectasis or immune deficiencies. Clinical symptoms vary in scope and intensity but commonly include chronic cough, often with purulent sputum while hemoptysis may also be present. Systemic symptoms include malaise, fatigue, and weight loss in advanced disease.

Current treatment of MAC-PD involves prolonged antibiotic therapy (frequently more than 18 months), with a combination of at least three antibiotics, including a rifamycin (rifampin or rifabutin), a macrolide (azithromycin or clarithromycin), ethambutol and/or injectable aminoglycosides (amongst others), which are associated with side-effects and a high failure rate. This treatment regimen is currently recommended by the American Thoracic Society (see American Journal of Respiratory and Critical Care Medicine Vol 175, 2007, page 367 by Griffith et al on behalf of the ATS Mycobacterial Diseases Subcommittee “An official ATS/IDSA Statement: Diagnosis, Treatment, and Prevention of Nontuberculous Mycobacterial Diseases”) and International Guidelines given the substantial in vitro and clinical activity displayed against MAC. Recently, amikacin liposome inhalation suspension (ALIS, Arikayce®) was approved by the US FDA for the treatment of MAC-PD in adults but otherwise this disease/condition has limited or no alternative treatment options. There are no other antibiotics approved for the treatment of MAC-PD and recommended use of the above agents is merely empirical.

Bedaquiline is a mycobacterium adenosine 5′-triphosphate (ATP) synthase inhibitor that has been developed as a part of a combination therapy for the treatment of pulmonary multidrug-resistant tuberculosis (MDR-TB) in adult patients. It has been approved for that indication under certain conditions under the tradename Sirturo® in territories including the US, Russia, the EU, Japan, South Africa and the Republic of Korea.

The marketed bedaquiline fumarate product Sirturo® is a tablet containing 100 mg of bedaquiline active ingredient. In the adult population, the first approval in Europe relates to the use of Sirturo® as a part of an appropriate combination regimen for pulmonary MDR-TB under certain conditions (when an effective treatment regimen cannot otherwise be composed for reasons of resistance or tolerability). Therein it is indicated (amongst other things) that Sirturo® should be used in combination with at least three medicinal products to which the patient's isolate has been shown to be susceptible in vitro. If in vitro testing results are unavailable, treatment may be initiated with Sirturo® in combination with at least four medicinal products to which the patient's isolate is likely to be susceptible. The product may also be administered by directly observed therapy (DOT). The recommended dosage is: (i) Weeks 1-2: 400 mg (4 tablets of 100 mg) once daily; (ii) Weeks 3-24: 200 mg (2 tablets of 100 mg) three times per week (with at least 48 hours between doses). The total duration of treatment with Sirturo® is 24 weeks. Other medicinal products that are used in combination may or should continue after completion of treatment with Sirturo®.

In the product Sirturo®, the active ingredient bedaquiline is in the form of a fumarate salt: (alpha S, beta R)-6-bromo-alpha-[2-(dimethylamino)ethyl]-2-methoxy-alpha-1-naphthalenyl-beta-phenyl-3-quinolineethanol, in particular (alpha S, beta R)-6-bromo-alpha-[2-(dimethylamino)ethyl]-2-methoxy-alpha-1-naphthalenyl-beta-phenyl-3-quinolineethanol (2E)-2-butenedioate (1:1) and may be represented by the following formula:

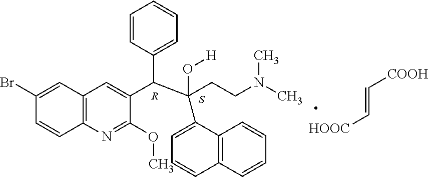

The fumarate salt can be prepared by reacting the corresponding free base with fumaric acid in the presence of a suitable solvent, such as for example isopropanol.

Bedaquiline is known to show activity against Mycobacteria including drug resistant strains, in particular Mycobacterium tuberculosis, M. bovis, M. avium, M. leprae, M. marinum, M. leprae, M. ulcerans, M. kansasii, and M. abscessus. The active ingredient, including salt thereof, shows activity against active, sensitive, susceptible Mycobacteria strains and latent, dormant, persistent Mycobacteria strains.

International patent application WO 2004/011436 first disclosed the activity of the free base of bedaquiline against Mycobacteria. Later documents such as international patent applications WO 2005/117875 and WO 2006/067048 disclose the further uses in the treatment of inter alia drug resistant tuberculosis and latent tuberculosis. International patent application WO 2008/068231 first described the suitability of the fumarate salt as a drug product indicating its acceptable bioavailability. The fumarate salt of bedaquiline is described as non-hygroscopic and stable. This document also discloses the preparation of certain formulations and tablets containing bedaquiline fumarate.

Given its in vitro activity in nontuberculous mycobacteria (especially in Mycobacterium abscessus and Mycobacterium avium), there have been reports that it has been used off-label as described in journal article Chest 2015; 148(2):499-506 by Philley et al “Preliminary Results of Bedaquiline as Salvage Therapy for Patients with Nontuberculous Mycobacterial Lung Disease”. This article indicates that bedaquiline has not been tested clinically for NTM disease and describes a small study of patients treated for 1-8 years, already on treatment at the start of bedaquiline therapy, and where 80% have macrolide-resistant isolates. Bedaquiline was administered according to the dosage as that used in TB trials, and in these studies, the patients were also receiving companion drugs (a mean of 5). It is stated that further study is clearly required to determine whether bedaquiline has a place in the management of NTM lung disease, and if so, to guide its appropriate use.

An abstract and certain results were also presented at a conference “Advances in the Management of Pulmonary NTM Disease” in San Diego in May 2018, where the topic was entitled “Macrolide Resistant Mycobacterium Avium Complex Lung Disease Treated with Bedaquiline” and it was described that patients (with macrolide resistant MAC lung disease) were administered with bedaquiline according to package guidelines in combination with companion drugs given at the discretion of two NTM pulmonary physicians. It was indicated that treatment options for macrolide resistant MAC lung disease were limited, and that bedaquiline used with companion therapy may be an option for drug resistant disease.

There is now provided a novel combination for clinical use in the treatment of a disease associated with NTM.

### DESCRIPTION OF THE INVENTION

The present disclosure provides a combination comprising (e.g. consisting of) bedaquiline, a macrolide (e.g. clarithromycin or azithromycin) and, optionally, ethambutol. Such combination is for use in the treatment of a disease associated with nontuberculous mycobacteria (NTM). In an embodiment, the combination comprises (e.g. consists of) bedaquiline, a macrolide (e.g. clarithromycin or azithromycin) and ethambutol. In an embodiment, such combinations are for clinical use (e.g. in a human subject), i.e. in vivo.

In an embodiment, there is provided a method of treating a disease associated with NTM in a patient, comprising administering to the patient an effective amount of a combination comprising (e.g. consisting of):
(i) bedaquiline;
(ii) a macrolide (e.g. clarithromycin or azithromycin); and
(iii) ethambutol.

In an embodiment, there is provided a method of treating a disease associated with NTM in a patient, comprising administering to the patient an effective amount of a combination comprising (e.g. consisting of):
(i) bedaquiline; and
(ii) a macrolide (e.g. clarithromycin or azithromycin).

These combinations mentioned herein are referred to herein as “the combinations of the invention”. As indicated above, the combinations of the invention comprise two or three active ingredients (bedaquiline, a macrolide and, optionally, ethambutol; in an embodiment, ethambutol is mandatory), which are active against mycobacteria, and specifically in this case, active against nontuberculous mycobacteria (especially Mycobacterium avium and Mycobacterium abscessus). Hence, these three components may be classed as anti-bacterials or antibiotics, and essentially they may act against the mycobacteria in a bacteriostatic (stopping the bacteria from reproducing but not necessarily killing them) or bacteridical (killing the bacteria) manner. In an embodiment, the combinations of the invention contain only these two or three active ingredients, although in an embodiment, such combinations may also contain an injectable aminoglycoside, for instance in severe cases of the mycobacterial infection or for those patients that do not respond to first-line oral therapy. In an embodiment, and in particular for certain patient populations (for instance, where it is either not needed or can be avoided), an injectable aminoglycoside is not employed. In the embodiment where it is employed, then the aminoglycoside may be any suitable one that has already received approval from a regulatory authority, for instance it may be a suitable one that has been approved by the US Food and Drug Administration (FDA) e.g. gentamicin, tobramycin, amikacin, plazomicin, streptomycin, neomycin, and/or paromomycin. In an embodiment, it is indicated that the combinations of the invention consist of two or three certain active ingredients (bedaquiline, a macrolide and, optionally, ethambutol; and in a further embodiment may further include an aminoglycoside), by which we mean that the combinations (or the method of treatment comprising administering such combinations to a patient) do not comprise any other active ingredients, such as compounds active against mycobacteria, compounds that are classed as anti-bacterials or antibiotics.

The essential components or antibacterial drugs of the combinations of the invention (i.e. bedaquiline, the macrolide and, in an embodiment, ethambutol) may be formulated separately (e.g. as defined herein) or may be formulated together. In an embodiment, such components (including bedaquiline, the macrolide and ethambutol) are formulated separately, for instance in the form in which they are marketed/commercially available (for existing approved indications).

In various embodiments (including the method of treating a disease associated with NTM in a patient), the antibacterial drugs of the combinations of the invention can be co-administered, in other embodiments the antibacterial drugs (of the combinations) may be sequentially administered, while in still other embodiments they can be administered substantially simultaneously. In some of the latter embodiments, administration entails taking such antibacterial drugs within 30 minutes or less of each other, in some embodiments 15 minutes or less of each other. In some embodiments, the antibacterial drugs are administered once per day, at approximately the same time each day. For example, the antibacterial drugs are administered within a time range of 4 hours of the original time of administration on the first day, that is, ±2 hours, or ±1 hour, or in still other embodiments ±30 minutes of the time on the original administration day. However, in an embodiment, the antibacterial drugs of the combinations of the invention (including bedaquiline, the macrolide and ethambutol) are administered in accordance with existing guidelines (e.g. in accordance with the regulatory label for the indication(s) for which the relevant active is approved).

In some embodiments, the antibacterial drugs of the combinations of the invention, or pharmaceutically acceptable salts thereof, are administered as separate oral capsules or oral tablets. Other formulations may include solid dispersions.

Bedaquiline can be used in its non-salt form or as a suitable pharmaceutically acceptable salt form, such as an acid addition salt form or base addition salt form.

The pharmaceutically acceptable acid addition salts are defined to comprise the therapeutically active non-toxic acid addition salt forms which bedaquiline is able to form. Said acid addition salts can be obtained by treating the free form of bedaquiline with appropriate acids, for example inorganic acids, for example hydrohalic acid, in particular hydrochloric acid, hydrobromic acid, sulfuric acid, nitric acid and phosphoric acid ; organic acids, for example acetic acid, hydroxyacetic acid, propanoic acid, lactic acid, pyruvic acid, oxalic acid, malonic acid, succinic acid, maleic acid, fumaric acid, malic acid, tartaric acid, citric acid, methanesulfonic acid, ethanesulfonic acid, benzenesulfonic acid, p-toluenesulfonic acid, cyclamic acid, salicyclic acid, p-aminosalicylic acid and pamoic acid. In particular, the fumarate salt is considered, given that this is the form employed in the already-marketed product Sirturo®.

Possible therapeutically active non-toxic base addition salt forms may be prepared by treatment with appropriate organic and inorganic bases. Appropriate base salts forms comprise, for example, the ammonium salts, the alkaline and earth alkaline metal salts, in particular lithium, sodium, potassium, magnesium and calcium salts, salts with organic bases, e.g. the benzathine, N-methyl-D-glucamine, hybramine salts, and salts with amino acids, for example arginine and lysine.

Conversely, said acid or base addition salt forms can be converted into the free forms by treatment with an appropriate base or acid.

The term addition salt as used in the framework of this application also comprises the solvates which bedaquiline as well as the salts thereof, are able to form. Such solvates are, for example, hydrates and alcoholates.

Whenever reference to bedaquiline is employed herein, we refer to the single stereoisomeric form that is employed in the marketed product Sirturo®, and which is disclosed in WO2004/011436 as an antimycobacterial agent.

The fumarate salt of the present invention can be prepared by reacting the corresponding free base with fumaric acid in the presence of a suitable solvent, such as for example isopropanol.

In a similar manner, the macrolide (e.g. clarithromycin or azithromycin) and the ethambutol may also be employed in their non-salt forms (or free forms) or in the form of a pharmaceutically acceptable salt. In an embodiment, the macrolide and ethambutol are in the forms in which they are already available/marketed.

For instance, bedaquiline may be administered as a tablet, e.g. formulated as the fumarate salt and containing 100 mg of the active ingredient bedaquiline. When the macrolide employed is clarithromycin, it may be administered as a 500 mg tablet (or, depending on the dose required and the patient, as a suspension, for instance the available suspension containing 250 mg/5 ml). Ethambutol may be administered (depending on the dose required) as a 100 mg or 400 mg tablet.

In an embodiment, the combinations of the invention are used in a particular treatment or administration regimen. For instance the method of treating a disease (associated with NTM) in a patient disclosed herein may have a particular treatment or administration regimen. Such treatment or administration regimen may comprise the following:
(i) bedaquiline: Weeks 1-2: 400 mg once daily (or “qd”); Weeks 3-24 (and optionally up to 52 weeks, i.e. Weeks 3-52): 200 mg three times per week (or “tiw”) (with at least 48 hours between doses);
(ii) the macrolide: for instance, when it is clarithromycin, 1000 mg per day, for instance 500 mg twice daily (i.e. 500 mg “bid”) and when it is azithromycin, 250 mg per day (alternatively clarithromycin may be administered at 500 mg per day, e.g. once daily; it may also be administered in accordance with local guidelines);
(iii) ethambutol: this will depend on the weight of the patient, and as per current guidelines the dosing will be 15 mg/kg per day (ethambutol may also be administered in accordance with local guidelines).

For instance, the administration regimens mentioned herein are applicable to the disease associated with NTM as defined/described hereinafter, and in particular relates to NTM-PD. The severity or type of the disease or the severity of the mycobacterial infection may also determine the dose or administration regime. In an embodiment, the American Thoracic Society (ATS) guidelines may be followed, for guidelines on administration of the macrolide (e.g. clarithromycin) and ethambutol, for the particular disease associated with NTM.

The total treatment regimen may be at least 24 weeks, for instance at least 32 weeks, e.g. about 48 weeks or about 52 weeks (however, in an embodiment, the treatment duration may last up to 18 months or even 24 months). In this respect, the dosing regime of bedaquiline is already indicated above for a possible 52-week period, and (if the duration runs to 18 or 24 months, then the dosing scheme for the Weeks 3-52 period will continue); similarly, the dosing of the macrolide (e.g. clarithromycin) and ethambutol will continue for the relevant period e.g. at least 24 weeks, at least 32 weeks, e.g. about 48 weeks or about 52 weeks (or, in a separate embodiment, up to 18 months or up to 24 months). In an embodiment, the treatment regime also comprises injectable aminoglycosides (e.g. in a situation where the ATS guidelines recommend this, for instance when the disease is severe; in this case e.g. a three times weekly injection may be administered). In an embodiment, the treatment regime does not comprise other drugs; however, companion drugs for instance to treat another disease (for instance which may already be being administered to the patient) may be tolerated (particularly when such drug is for a disease other than a bacterial infection, and e.g. its drug-drug interaction with the essential antibacterials of the combination of the invention has already been studied) although, in an embodiment, any other drugs are not administered during the treatment regimens described herein.

In an embodiment, the macrolide employed in the combinations of the invention is clarithromycin.

In an embodiment, the bedaquiline is administered after food, as that may increase the bioavailability of the drug.

In an embodiment, dosing of the macrolide (e.g. clarithromycin) and ethambutol will be as per local guidelines.

In an embodiment, the antibacterial drugs of the combination of the invention are taken orally, with administration occurring at approximately the same time each day.

All amounts mentioned in this disclosure refer to the free form (i.e. non-salt form). The values given below represent free-form equivalents, i.e., quantities as if the free form would be administered. If salts are administered the amounts need to be calculated in function of the molecular weight ratio between the salt and the free form

The daily doses described herein are calculated for an average body weight of about 70 kg and should be recalculated in case of paediatric applications, or when used with patients with a substantially diverting body weight.

It is indicated herein that the combinations used herein are useful in the treatment of a disease associated with nontuberculous mycobacteria (NTM). A method of treatment is also described herein, which relates to treatment of a disease associated with NTM in a patient, and the patient is administered an effective amount of a combination of the invention.

As used herein the term “disease associated with NTM in a patient” refers to a patient (or subject, e.g. human patient) being infected with a nontuberculous mycobacteria (especially Mycobacterium abscessus and Mycobacterium avium). In particular such disease may be a pulmonary disease that is caused by NTM, and thus in an embodiment the disease is NTM-PD. NTM-PD is distinct from the pulmonary infection caused by M. tuberculosis, for which bedaquiline is currently indicated. Mycobacterium avium is part of the Mycobacterium avium complex (MAC) which accounts for up to 70% of NTM-positive sputum cultures (although there are regional differences) and is one of the three NTM-PD species most commonly implicated in human disease in North America. MAC are naturally-occurring organisms common in water and soil, often colonizing natural water sources such as indoor water systems, hot tubs and pools. MAC pulmonary disease (MAC-PD) is most often seen in post-menopausal women and patients with underlying lung disease such as cystic fibrosis, bronchiectasis or immune deficiencies. Clinical symptoms vary in scope and intensity but commonly include chronic cough, often with purulent sputum while hemoptysis may also be present. Systemic symptoms include malaise, fatigue, and weight loss in advanced disease.

Included within NTM-PD are treatment-refractory NTM-PD patients, and, as indicated above, the most common NTM-PD is MAC-PD. Hence, in an embodiment, when the term “disease associated with NTM” is referred to herein, this refers to NTM-PD in general, and in a further embodiment, it refers to NTM-PD in treatment-refractory patients; in a further embodiment, it refers to MAC-PD and in a yet further embodiment it refers to MAC-PD in treatment-refractory patients. Treatment-refractory MAC-PD patients are defined as patients who are sputum culture positive for MAC after a minimum of 6 months of guideline-based therapy for MAC-PD infection. Refractory patients treated with the current standard of care have very poor clinical outcomes with approximately 10% ultimately culture converting after twelve months of therapy even with intensification of treatment and the use of aminoglycosides. This patient population therefore also represents an area of unmet medical need. In an embodiment, the disease associated with NTM (e.g. NTM-PD, such as MAC-PD) is accompanied with an underlying lung disease (such as cystic fibrosis, or another as mentioned herein).

Due to its novel mode of action (inhibition of ATP synthase) bedaquiline defines a new class of anti-TB compounds and currently, no other drugs belonging to the same pharmacological class are available, thus minimising the potential for cross-resistance. The combinations of the invention described herein thus have an advantage that bedaquiline is a component thereof.

As used herein, “effective amount” refers to the amount of each of the components of the combinations of the invention, or any pharmaceutically acceptable salts thereof, that elicits the biological or medicinal response in a tissue system (e.g., blood, plasma, biopsy) or warm-blooded animal (e.g., human), that is being sought by a health care provider, which includes alleviation of the symptoms of the disease being treated.

Patients treated according to the methods of the disclosure can be “first-line” patients. As used herein, this refers to the patient not having previously received treatment with any drug—investigational or approved—for the disease to be treated (associated with NTM). In a further embodiment, the patients to be treated are not first-line patients, but patients that have already received treatment, for instance patients that have been diagnosed with the disease, still testing positive after 6 months of other guideline therapy (i.e. tested positive in sputum culture for MAC after a minimum of 6 months of guideline-based therapy). Hence, in an embodiment, the patients are treatment-refractory patients or salvage patients. In a further embodiment, the isolates of the NTM are not macrolide-resistant. However, in a further embodiment, when the isolates of the NTM are macrolide-resistant, this may result in a combination of bedaquiline and ethambutol (without a macrolide), in which case the combination of actives may only consist of those two drugs (although optionally in this embodiment a further, different antibacterial may be added, which is not a macrolide).

To date, the surrogate markers predictive of clinical treatment response have not been defined. The current primary endpoint for “treatment” is sputum culture conversion, defined as 3 consecutive negative monthly sputum cultures by the 6 months timepoint after start of treatment. The primary efficacy outcome time point is selected at 6 months because the majority of the microbiological response occurred during this time-period in a recently completed trial ALIS, for instance as described in American Journal of Respiratory and Critical Care Medicine Volume 195 Number 6, Mar. 15, 2017 “Randomized Trial of Liposomal Amikacin for Inhalation in Nontuberculous Mycobacterial Lung Disease” by Griffith et al, and in a AJRCCM article published 14 Sep. 2018 “Amikacin Liposome Inhalation Suspension for Treatment-Refractory Lung Disease Caused by Mycobacterium avium Complex (CONVERT): A Prospective, Open-Label, Randomized Study” by Griffith et al.

As mentioned herein, the combination of antibacterial drugs as described herein may be co-administered, sequentially administered, or administered substantially simultaneously (as described herein). Hence the individual dosage forms of each of the antibacterial drugs can be administered as separate forms (e.g. as separate tablets or capsules) as described herein.

In an embodiment, there is provided a process for preparing a combination product as defined herein comprising:
bringing into association each of the components (e.g. as separate pharmaceutical formulations) of the combination product and co-packaging (e.g. as a kit of parts) or indicating that the intended use is in combination (with the other components); and/or
bringing into association each of the components in the preparation of a pharmaceutical formulation comprising such components.

In current MAC-PD regimens where a rifamycin is combined with clarithromycin, it is reported that exposure to clarithromycin is suboptimal due to induction of metabolism by the rifamycin component, for instance as described in Journal of Pharmaceutical Health care and Sciences (2015) 1:32 by Shimomura et al “Serum concentrations of clarithromycin and rifampicin in pulmonary Mycobacterium avium complex disease: long term changes due to drug interactions and their association with clinical outcomes”. The combinations of the invention may overcome this. The combinations of the invention may also have the advantage that they are more efficacious than, have a better safety profile and/or have fewer side effects than those treatment regimens already own or already recommended (e.g. by the ATS).

The following examples are merely illustrative and are not intended to limit the disclosure to the materials, conditions, or process parameters set forth therein

### EXAMPLES

### Reference Example 1

### In Vitro Activity of Bedaquiline

Bedaquiline has a unique spectrum in its specificity to mycobacteria, including atypical species important in humans such as M. avium, M. kansasii, and the fast growers M. fortuitum and M. abscessus. M. avium, M. kansasii and M. abscessus can be responsible for causing NTM disease.

Bedaquiline minimum inhibitory concentration (MIC) ranges for M. tuberculosis were ≤0.008 μg/ml to 0.12 μg/ml regardless of resistance sub-type. Bedaquiline MICs were generally <0.1 μg/ml for other mycobacterial species, including species naturally resistant to many other anti-TB agents and involved in opportunistic infections, such as M. avium, M. abscessus. M. fortuitum and M. marinum. In comparison to M. tuberculosis, higher MICs were found for 1 isolate each of M. abscessus (0.25 μg/ml) and M. ulcerans (0.50 μg/ml) (see the table below). The activity of bedaquiline appeared to be specific for Mycobacterium species.

| | | Bedaquiline MIC (μg/ml) | |
|---|---|---|---|
| Mycobacterial Organism | n | MIC range | Median |
| M. bovis | 1 | — | 0.003 |
| M. avium/M. | 7 | 0.007-0.010 | 0.010 |
| intracellulare (MAC) | | | |
| M. kansasii | 1 | — | 0.003 |
| M. marinum | 1 | — | 0.003 |
| M. fortuitum | 5 | 0.007-0.010 | 0.010 |
| M. abscessus | 1 | — | 0.250 |
| M. smegmatis | 7 | 0.003-0.10 | 0.007 |
| M. ulcerans | 1 | — | 0.500 |

### Example 1

### Further In Vitro Testing Against Slow Grower Nontuberculous Mycobacteria (NTM)

Objective

To determine the Minimum Inhibitory Concentration (MIC) and the Minimum Bactericidal Concentration (MBC) of bedaquiline against a clinical isolate of NTM, the most common NTM respiratory pathogens using the resazurin microtiter assay (REMA), as per the following article by Martin A et al “Resazurin microtiter assay plate testing of Mycobacterium tuberculosis susceptibilities to second-line drugs: rapid, simple, and inexpensive method. AAC, 2003 November; 47 (11):3616-9.

Methodology

In the REMA plate, the concentration rage of bedaquiline was from 2 to 0.0035 μg/ml. Each experiment was performed in triplicate in 7H9 medium supplemented with OADC and glycerol. Plates were sealed in plastic bags and incubated at 37° C. for 7 days. After 7 days incubation, 30 μl of the resazurin 0.01% was added to all the wells and the plate again sealed and incubated overnight for colour development.

MIC was interpreted as the lowest concentration of the bedaquiline that prevents a change in colour of the resazurin. MIC values were scored for each NTM species. The positive control (growth control positive=medium+bacteria) should show positive growth and the negative control (or sterile control, containing only medium) should show no growth within the incubation period. (There is also a bedaquiline control, consisting of bedaquiline+medium only.)

MBC Determination by Resazurin Microtiter Assay (REMA)

The MBC test allows determination of the minimum concentration of an agent necessary to achieve a bactericidal effect. The MBC was determined once the MIC was determined previously. To perform the MBC, the dilution representing the MIC and at least two of the more concentrated test product dilutions were plated and enumerated to determine viable CFU/ml. MBC is the lowest concentration at which bedaquiline demonstrated bactericidal activities against a particular NTM species.

Strain for Quality Control

Mycobacterium xenopi was used for quality control as the MIC for bedaquiline, because this species is known to be naturally resistant to bedaquiline (as described in the journal article by Andries K et al: “A diarylquinoline drug active on the ATP synthase of Mycobacterium tuberculosis” in Science, 2005 Jan. 14; 307(5707): 223-7). This strain was tested each time a new lot, medium, drug was prepared.

Slow Grower Mycobacteria Proposed to be Tested

NTM clinical isolates used for this study were isolated from patients. In total, there were 18 isolates (in addition to the control strain): mycobacterium avium (x4 isolates), mycobacterium intracellulare (x4), mycobacterium chimaera (x3), mycobacterium kansasii (x2), mycobacterium ulcerans (x2), mycobacterium simiae (x2) and mycobacterium marinum (x1).

Summary of MIC and MBC Results

MIC and MBC ranges were determined for the 19 NTM slow growers tested

| Slow grower | | |
|---|---|---|
| mycobacteria (n = | Bedaquiline | |
| number of isolates) | MIC (μg/ml) | MBC (μg/ml) |
| Control Strain | | |
| Mycobacterium | >2 | ND |
| xenopi (×1) | | |
| Clinical Isolates | | |
| Mycobacterium | 0.007 | >2 |
| avium (×4) | 0.015 | >2 |
| | 0.007 | 1 |
| | 0.015 | >2 |
| Mycobacterium | 0.015 | 2 |
| intracellulare (×4) | 0.015 | 1 |
| | 0.015 | 1 |
| | 0.007 | 1 |
| Mycobacterium | 0.007 | 1 |
| chimaera (×3) | 0.015 | 2 |
| | 0.015 | 1 |
| Mycobacterium | 0.015 | 1 |
| kansasii (×2) | 0.015 | 0.03 |
| Mycobacterium | ND | |
| ulcerans (×2) | | |
| Mycobacterium | 0.015 | 1 |
| simiae (×2) | 0.03 | >2 |
| Mycobacterium | 0.015 | 2 |
| marinum (×1) | | |

ND = not determined

Bedaquiline showed bactericidal activity for the majority of clinical isolates tested. MBC was considered the lowest concentration at which the bedaquiline kills 100% of the bacteria.

### Example 2

### In Vivo Testing

Objectives

Primary Objective

The primary objective is to assess the efficacy of bedaquiline plus a macrolide (clarithromycin) and ethambutol (bedaquiline/clarithromycin/ethambutol) compared with a rifamycin plus a macrolide (clarithromycin) and ethambutol (rifamycin/clarithromycin/ethambutol) for the treatment of NTM-PD in adult patients with treatment-refractory NTM-PD due to MAC.

Secondary Objectives

The secondary objectives are in adult patients with treatment-refractory NTM-PD due to MAC to:
To evaluate changes in quantitative sputum Colony Forming Units (CFU) counts over a 3- and 6-month treatment period with: bedaquiline/clarithromycin/ethambutol compared to rifamycin/clarithromycin/ethambutol.
To evaluate sputum culture negativity at 1, 2, 3, 4 and 5 months during treatment and at the end of 3-month follow-up after 12 months of treatment.
To evaluate sputum culture conversion after 12 months of treatment.
To evaluate the proportion of subjects who acquire resistance to clarithromycin post-baseline.
To evaluate the proportion of subjects who acquire resistance to bedaquiline (at least 4-fold increase in bedaquiline MIC) compared to baseline.
To evaluate the safety and tolerability of treatment with bedaquiline/clarithromycin/ethambutol compared to rifamycin/clarithromycin/ethambutol.
To evaluate the percentage of patients that deviate from the protocol including those for whom therapy is modified.
To evaluate the changes in patient-reported health status after 6 and 12 months of treatment with bedaquiline/clarithromycin/ethambutol compared to rifamycin/clarithromycin/ethambutol.
To evaluate the changes in lung function parameters: forced expiratory volume in 1 second (FEV1 [L]), forced vital capacity (FVC [L]), inspiratory capacity (IC [L]), functional residual capacity (FRC [L]), total lung capacity (TLC [L]) after 6 and 12 months of treatment with bedaquiline/clarithromycin/ethambutol compared to rifamycin/clarithromycin/ethambutol.
To evaluate the changes in 6-minute walking distance (6MWD) after 6 and 12 months of treatment with bedaquiline/clarithromycin/ethambutol compared to rifamycin/clarithromycin/ethambutol.
To evaluate the pharmacokinetics of bedaquiline and clarithromycin as part of the proposed NTM regimen.
To evaluate the pharmacokinetic-pharmacodynamic relationships for safety and efficacy of bedaquiline as part of the proposed NTM regimen.
To evaluate long-term safety and tolerability of bedaquiline over 120 weeks post-baseline.

Endpoints

Primary Endpoint

Sputum culture conversion (defined as 3 consecutive negative monthly sputum cultures) by the 6 months timepoint after start of investigational treatment.

Secondary Endpoints

The secondary endpoints are:
The changes in quantitative sputum CFU counts (solid cultures) over a 3- and 6-month treatment period with bedaquiline/clarithromycin/ethambutol compared to rifamycin/clarithromycin/ethambutol.
Sputum culture negativity (liquid cultures) at 1, 2, 3, 4 and 5 months during treatment.
Sputum culture conversion (liquid cultures) after 12 months of treatment.
Sputum culture negativity (liquid cultures) at the end of 3-month follow-up after 12 months of treatment.
The proportion of subjects who acquire resistance to clarithromycin post-baseline.
The proportion of subjects who acquire resistance to bedaquiline (at least 4-fold increase in bedaquiline MIC) compared to baseline.
Safety and tolerability (including survival follow-up to 120 weeks post-baseline).
The percentage of patients that deviate from the protocol including those for whom therapy is modified.
The differences in patient-reported health status after 6 and 12 months of treatment (SGRQ).
The differences in lung function parameters: FEV1 (L), FVC (L), IC (L), FRC (L), TLC (L), after 6 and 12 months of treatment.
The differences in 6-minute walking distance after 6 and 12 months of treatment (6MWD).
Pharmacokinetic parameters (by population pharmacokinetic analysis) for bedaquiline and clarithromycin.
PK/PD relationships for safety and efficacy of bedaquiline.

Study Design

This is a multicenter, randomized, open-label, active-controlled, Phase 2a study to evaluate the efficacy of bedaquiline plus a macrolide (clarithromycin) and ethambutol versus a rifamycin plus a macrolide (clarithromycin) and ethambutol in the treatment of adult patients with treatment-refractory NTM-PD due to MAC.

Adult participants with treatment-refractory NTM-PD due to MAC (defined as patients who are sputum culture positive for MAC after a minimum of 6 months of guideline-based therapy) will be enrolled. In an embodiment, subjects with fibro-cavitary NTM-PD and cystic fibrosis will be excluded.

Participants who meet all the eligibility criteria will be randomized in a 1:1 ratio to receive 1 of the following 2 treatment regimens:
Treatment Group A: Rifamycin*+clarithromycin (e.g. 500 mg per day or 1000 mg per day)+ethambutol 15 mg·kg/day (maximum daily dose of 1600 mg)
Treatment Group B: Bedaquiline**+clarithromycin (e.g. 500 mg per day or 1000 mg per day)+ethambutol 15 mg·kg/day (maximum daily dose of 1600 mg)

* participants may receive either rifabutin or rifampin determined by the preference of the treating physician. Rifabutin will be dosed at 150 mg for subjects weighing <50 kg or 300 mg for subjects weighing ≥50 kg. Rifampin will be dosed at 10 mg/kg/day up to a maximum dose of 600 mg.

** participants will be dosed with bedaquiline as follows:
Weeks 1-2: 400 mg (4 tablets of 100 mg) qd.
Weeks 3-52: 200 mg (2 tablets of 100 mg) tiw (with at least 48 hours between doses).

Subjects will be randomly assigned to 1 of two treatment groups based on a computer-generated randomization schedule prepared before the study by or under the supervision of the Sponsor. The randomization will be balanced by using randomly permuted blocks.

All study drugs will be taken orally, drug administration should occur at approximately the same time each day.

Dosing of rifamycin, clarithromycin and ethambutol will be as per local guidelines.

The study will consist of a screening period (1 month), baseline visit (Day 1), an open-label treatment period of 12 months (Day 1 to Week 48), and a follow-up period of 3 months (Week 48 to Week 60). The entire study duration for each subject will be 15 months. Participants will return for study visits biweekly in the first 3 months, and at week 16, 20, 24, 32, 40, 48 and 60 thereafter.

All subjects will be followed until 120 weeks post-baseline to collect long-term safety and tolerability, pharmacokinetics, MAC treatment outcomes, and anti-mycobacterial information. Subjects who prematurely discontinue from study drug and study procedures will be followed up for survival until 120 weeks post-baseline, unless they withdraw from the study (i.e., withdraw consent/assent). The total study duration (including the treatment and follow-up phases, but excluding the screening phase) will be 120 weeks for each participant. The study is considered completed with the last visit of the last participant participating in the study.

In order to improve bioavailability of bedaquiline, this should be administered with food as this improves by approximately 2-fold.

Sample Size Determination (Will be Determined)

The primary endpoint is sputum culture conversion after 6 months of therapy. The sample size will be determined based on e.g. the response rates of historical controls and the results of a clinical trial of ALIS in an analogous population. Based on this, the total number of subjects (and subjects per arm) to be enrolled is determined.

Statistical Analyses

The primary analysis in this study will be performed when subjects have reached month 6 after start of investigational treatment or have discontinued earlier. The primary endpoint is sputum culture conversion (defined as 3 consecutive negative monthly sputum cultures) by the 6 months timepoint after start of investigational treatment. In addition to sputum culture conversion, drug susceptibility testing, effects of bedaquiline on clinical course endpoints, next to endpoints related to safety, and PK, will be analyzed to support early Phase 3 preparations, including regulatory interactions.

MAC-PD Treatment Outcome Analyses

The Mantel-Haenszel test will be used to compare culture conversion rate at 6 months (primary endpoint). The same test will be used to compare the proportion of patients who are culture negative at other timepoints (including 1, 2, 4, 6 and 12 months). The Kaplan-Meier method will be used to estimate the proportion of subjects achieving culture conversion over the 12 months treatment period, and difference between treatment arms will be compared using log-rank test. A key microbiological endpoint is decline in bacterial load quantified by CFU. This is exploratory and to our knowledge there are very few data on the early microbiological activity in patients with NTM. Janssen will compare the change from baseline in the median log₁₀ CFU count out to three months and at intervening time points using a Wilcoxon rank sum test. Safety analysis will involve descriptive summary of frequency of adverse events, summary of significant change in laboratory values, ECG parameters, and vital signs by time point.

The invention may be described by the following clauses (or “clauses of the invention”).

1. A combination comprising (e.g., consisting of) bedaquiline, a macrolide (e.g., clarithromycin or azithromycin) and ethambutol.

2. A combination as described in claim 1 for use in the treatment of a disease associated with nontuberculous mycobacteria (NTM).

3. A method of treating a disease associated with NTM in a patient, comprising administering to the patient an effective amount of a combination comprising (e.g., consisting of):
(i) bedaquiline;
(ii) a macrolide (e.g., clarithromycin or azithromycin); and
(iii) ethambutol.

4. A combination for use as described in claim 1 or claim 2, which is administered in a particular regimen, or, a method as described in claim 3, where the administering to the patient consists of a particular regimen, wherein (in each case), the regimen comprises:
administration of bedaquiline: Weeks 1-2: 400 mg once daily (or “qd”); Weeks 3-24 (and optionally up to 52 weeks, i.e., Weeks 3-52): 200 mg three times per week (or “tiw”) (with at least 48 hours between doses).

5. A combination or method of claim 4, wherein the regimen comprises:
administration of the macrolide: for instance, when it is clarithromycin, 1000 mg per day, for instance 500 mg twice daily (i.e., 500 mg “bid”) and when it is azithromycin, 250 mg per day.

6. A combination or method as described in claim 4 or claim 5, wherein the regimen comprises:
administration of ethambutol: using the dosing 15 mg/kg per day.

7. A combination or method as described in any one of claims 4 to 6, wherein the total treatment regimen is about 52 weeks.

8. A combination or method as described in any one of claims 4 to 7, wherein the treatment regimen does not comprise any other drugs.

9. A combination or method as described in any one of the preceding clauses, wherein the disease associated with nontuberculous mycobacteria (NTM) is NTM pulmonary disease (NTM-PD).

10. A combination or method as described in claim 9, wherein the disease is NTM-PD in which the isolates of the NTM are not macrolide-resistant.

11. A combination as described in any one of claims 1, 2 or 4 to 10, wherein the combination of antibacterial drugs as described herein may be co-administered, sequentially administered, or administered substantially simultaneously.

12. A process for preparing a combination as described in claim 11, which comprises:
bringing into association each of the components (e.g., as separate pharmaceutical formulations) of the combination product and co-packaging (e.g., as a kit of parts) or indicating that the intended use is in combination (with the other components); and/or
bringing into association each of the components in the preparation of a pharmaceutical formulation comprising such components.
